(19) United States
(12) Patent Application Publication
(10) Pub. No.: US 2013/0023984 A1
(43) Pub. Date: Jan. 24, 2013

(54) COMMISSURE MODIFICATION OF PROSTHETIC HEART VALVE FRAME FOR IMPROVED LEAFLET ATTACHMENT

(71) Applicant: Brian S. Conklin, Orange, CA (US)

(72) Inventor: Brian S. Conklin, Orange, CA (US)

(73) Assignee: EDWARDS LIFESCIENCES CORPORATION, Irvine, CA (US)

(21) Appl. No.: 13/552,520

(22) Filed: Jul. 18, 2012

Related U.S. Application Data
(60) Provisional application No. 61/509,889, filed on Jul. 20, 2011.

(51) Int. Cl. A61F 2/24

(52) U.S. Cl. 623/2.14

(57) ABSTRACT

Embodiments of the present disclosure provide an improved support frame for a prosthetic heart valve. The support frame can include a plurality of diamond-shaped cells arranged in a plurality of circumferential rows. Three cells corresponding to the leaflet commissures can be configured as commissure tip cells, with elongated rounded portions rather than a diamond shape. The commissure tip cells can allow for insertion of leaflet tabs, thereby allowing the leaflets to be sutured outside of the valve. In this manner, the leaflet sutures can be removed from areas of high stress during physiologic loading. Thus, currently disclosed embodiments of a support frame can allow for use of thinner leaflet materials than possible with conventional prosthetic heart valves, without sacrificing valve durability in some embodiments.

### RELATED APPLICATIONS

The present application claims priority under 35 U.S.C. §119(e) to U.S. provisional application No. 61/509,889 filed Jul. 20, 2011.

### FIELD

The present invention concerns embodiments of a prosthetic heart valve frame.

### BACKGROUND

Prosthetic cardiac valves have been used for many years to treat cardiac valvular disorders. The native heart valves (such as the aortic, pulmonary and mitral valves) serve critical functions in assuring the forward flow of an adequate supply of blood through the cardiovascular system. These heart valves can be rendered less effective by congenital, inflammatory or infectious conditions. Such damage to the valves can result in serious cardiovascular compromise or death. For many years the definitive treatment for such disorders was the surgical repair or replacement of the valve during open heart surgery, but such surgeries are prone to many complications. More recently a transvascular technique has been developed for introducing and implanting a prosthetic heart valve using a flexible catheter in a manner that is less invasive than open heart surgery.

In this technique, a prosthetic valve is mounted in a crimped state on the end portion of a flexible catheter and advanced through a blood vessel of the patient until the valve reaches the implantation site. The valve at the catheter tip is then expanded to its functional size at the site of the defective native valve such as by inflating a balloon on which the valve is mounted. Alternatively, the valve can have a resilient, self-expanding stent or frame that expands the valve to its functional size when it is advanced from a delivery sheath at the distal end of the catheter.

Balloon-expandable valves typically are preferred for replacing calcified native valves because the catheter balloon can apply sufficient expanding force to anchor the frame of the prosthetic valve to the surrounding calcified tissue. On the other hand, self-expanding valves typically are preferred for replacing a defective, non-stenotic (non-calcified) native valve. One drawback associated with implanting a self-expanding valve is that as the operator begins to advance the valve from the open end of the delivery sheath, the valve tends to “jump” out very quickly from the end of the sheath; in other words, the outward biasing force of the valve's frame tends to cause the valve to be ejected very quickly from the distal end of the delivery sheath, making it difficult to deliver the valve from the sheath in a precise and controlled manner and increasing the risk of trauma to the patient.

Another problem associated with implanting a percutaneous prosthetic valve in a non-stenotic native valve is that the prosthetic valve may not be able to exert sufficient force against the surrounding tissue to resist migration of the prosthetic valve. Typically, the stent of the prosthetic valve must be provided with additional anchoring or attachment devices to assist in anchoring the valve to the surrounding tissue. Moreover, such anchoring devices or portions of the stent that assist in anchoring the valve typically extend into and become fixed to non-diseased areas of the vasculature, which can result in complications if future intervention is required, for example, if the prosthetic valve needs to be removed from the patient.

U.S. patent application Ser. No. 12/429,040 (referred to herein as “the '040 Application”), filed Apr. 23, 2009, which is incorporated herein by reference, discloses embodiments of a prosthetic heart valve and delivery apparatus designed to address these and other issues in the prior art. In one embodiment disclosed in the '040 Application, a self-expanding valve comprises an expandable stent that is shaped to maintain the valve in the aortic annulus against axial movement without anchors or retaining devices that engage the surrounding tissue. A delivery apparatus for delivering a self-expanding prosthetic valve can be configured to allow controlled and precise deployment of the valve from a valve sheath so as to minimize or prevent jumping of the valve from the valve sheath.

FIGS. 1-2 illustrate one embodiment of a prior art support frame 12 for a prosthetic heart valve as disclosed in the '040 Application. Generally, the '040 Application discloses a prosthetic aortic heart valve having a self-expandable support frame 12. The frame 12 comprises repeating, identical diamond-shaped cells at every position around the frame, with multiple rows (e.g., three rows, as shown in FIGS. 1-2) of such cells. The valve is radially compressible to a compressed state for delivery through the body to a deployment site and expandable to its functional size at the deployment site.

The support frame or stent 12 supports a flexible leaflet section, but is shown without the leaflet section for purposes of illustration. As shown, the stent 12 can be formed from a plurality of longitudinally extending, generally sinusoidal shaped frame members, or struts, 16. The struts 16 are formed with alternating bends and are welded or otherwise secured to each other at nodes 18 formed from the vertices of adjacent bends so as to form a mesh structure. The struts 16 can be made of a suitable shape memory material, such as the nickel titanium alloy known as Nitinol, that allows the valve to be compressed to a reduced diameter for delivery in a delivery apparatus and then causes the valve to expand to its functional size inside the patient's body when deployed from the delivery apparatus. If the valve is a balloon-expandable valve that is adapted to be crimped onto an inflatable balloon of a delivery apparatus and expanded to its functional size by inflation of the balloon, the stent 12 can be made of a suitable ductile material, such as stainless steel.

The stent 12 has an inflow end 26 and an outflow end 27. The mesh structure formed by struts 16 comprises a generally cylindrical “upper” or outflow end portion 20, an outwardly bowed or distended intermediate section 22, and an inwardly bowed “lower” or inflow end portion 24. The intermediate section 22 desirably is sized and shaped to extend into the Valsalva sinuses in the root of the aorta to assist in anchoring the valve in place once implanted. As shown, the mesh structure desirably has a curved shape along its entire length that gradually increases in diameter from the outflow end portion 20 to the intermediate section 22, then gradually decreases in diameter from the intermediate section 22 to a location on the inflow end portion 24, and then gradually increases in diameter to form a flared portion terminating at the inflow end 26.

When the valve is in its expanded state, the intermediate section 22 has a diameter D₁, the inflow end portion 24 has a minimum diameter D₂, the inflow end 26 has a diameter D₃, and the outflow end portion 20 has a diameter D₄, where D₂ is less than D₁ and D₃ and D₄ is less than D₂. In addition, D₁ and D₃ desirably are greater than the diameter than the native annulus in which the valve is to be implanted. In this manner, the overall shape of the stent 12 assists in retaining the valve at the implantation site. More specifically, the valve can be implanted within a native valve (the aortic valve in the illustrated example) such that the lower section 24 is positioned within the aortic annulus, the intermediate section 24 extends above the aortic annulus into the Valsalva's sinuses, and the lower flared end 26 extends below the aortic annulus. The valve is retained within the native valve by the radial outward force of the lower section 24 against the surrounding tissue of the aortic annulus as well as the geometry of the stent. Specifically, the intermediate section 24 and the flared lower end 26 extend radially outwardly beyond the aortic annulus to better resist against axial dislodgement of the valve in the upstream and downstream directions (toward and away from the aorta). Depending on the condition of the native leaflets, the valve typically is deployed within the native annulus with the native leaflets folded upwardly and compressed between the outer surface of the stent 12 and the walls of the Valsalva sinuses.

Known prosthetic valves having a self-expanding frame typically have additional anchoring devices or frame portions that extend into and become fixed to non-diseased areas of the vasculature. Because the shape of the stent 12 assists in retaining the valve, additional anchoring devices are not required and the overall length L of the stent can be minimized to prevent the stent upper portion 20 from extending into the non-diseased area of the aorta, or to at least minimize the extent to which the upper portion 20 extends into the non-diseased area of the aorta.

However, the frame design shown in FIGS. 1-2 does not allow for optimal leaflet attachment methods. For example, the diamond-shaped cells of the frame 12 do not provide a way to shield leaflet sutures from the high stresses imparted on the leaflets during physiologic opening and closing of the valve. Because of the leaflet attachment methods required by the diamond-shaped cells disclosed of the frame 12, the leaflet sutures can experience high tension during valve closing and can tear through the leaflets after repeated cycling of the valve. The high stresses experienced by the leaflets during use limit the valve's durability and life span, especially when thin tissue such as porcine pericardium is used as the leaflet material. On the other hand, the use of such thin leaflet materials is desirable in order to minimize the crimped diameter of the prosthetic valve for easier delivery to the implantation site. There thus remains a need for an improved stent frame for use with a prosthetic valve, such as the valve disclosed in the '040 Application.

### SUMMARY

Certain embodiments of the present disclosure provide a frame for use with a prosthetic heart valve that can reduce forces experienced by the leaflets and leaflet sutures during physiologic loading. In one embodiment, three cells positioned adjacent the outflow end of the frame and corresponding to the commissures can include a rounded projection, so as to form three commissure tip cells, those commissure tip cells being distinct from the other frame cells. The commissure tip cells can allow for use of leaflets having tabs on opposing ends that can be arranged such that the tabs of adjacent leaflets pass through the commissure tip cell and be sutured together outside of the valve. In this manner, the leaflet sutures are removed from the high-stress commissure area, and thereby shielded from said high stresses, thus reducing the risk of tearing through the leaflet material. Disclosed embodiments of a frame for use with a prosthetic heart valve such as the valve disclosed in the '040 Application can therefore be optimally configured for use with thin leaflet materials, such as porcine pericardium or other leaflet material.

In some embodiments, the leaflet tabs can be wrapped around a bar, pin, or other small component or insert positioned outside of the support frame. Further, the leaflet tabs can be sutured around the bar, pin, or other small component. Thus, the leaflet sutures can be positioned outside of the support frame, and away from the high stress commissure area. In some embodiments, at least a portion of the support frame can be cloth-covered, to facilitate leaflet attachment outside of the support frame.

The foregoing and other features and advantages of the invention will become more apparent from the following detailed description, which proceeds with reference to the accompanying figures.

### BRIEF DESCRIPTION OF THE DRAWINGS

FIG. 1 is a side elevation view of a prior art support frame for a prosthetic valve that can be used to replace the native aortic valve of the heart.

FIG. 2 is a perspective view of the prior art support frame of the valve of FIG. 1.

FIG. 3 is a perspective view of a support frame for a prosthetic valve according to the present disclosure.

FIG. 4 is a perspective view of the support frame of FIG. 3 with leaflet tabs positioned through the commis sure tip cells.

FIG. 5 shows a perspective view of the support frame of FIG. 4, with the leaflet tabs secured around a pin.

### DETAILED DESCRIPTION

As used in this application and in the claims, the singular forms “a,” “an,” and “the” include the plural forms unless the context clearly dictates otherwise. Additionally, the term “includes” means “comprises.” Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Disclosed embodiments of an improved radially expandable and compressible support frame can be used with any prosthetic valve, such as a prosthetic aortic heart valve. Embodiments of the improved support frame can advantageously allow for the leaflet sutures to be positioned outside of the valve, thereby removing the leaflet sutures from areas of high stress during physiologic loading. Positioning the leaflet sutures outside of the valve (e.g., outside of the support frame, rather than securing the leaflets to the support frame itself), can allow for use of thinner leaflet materials, which in turn can allow for a smaller crimped delivery diameter of the prosthetic valve.

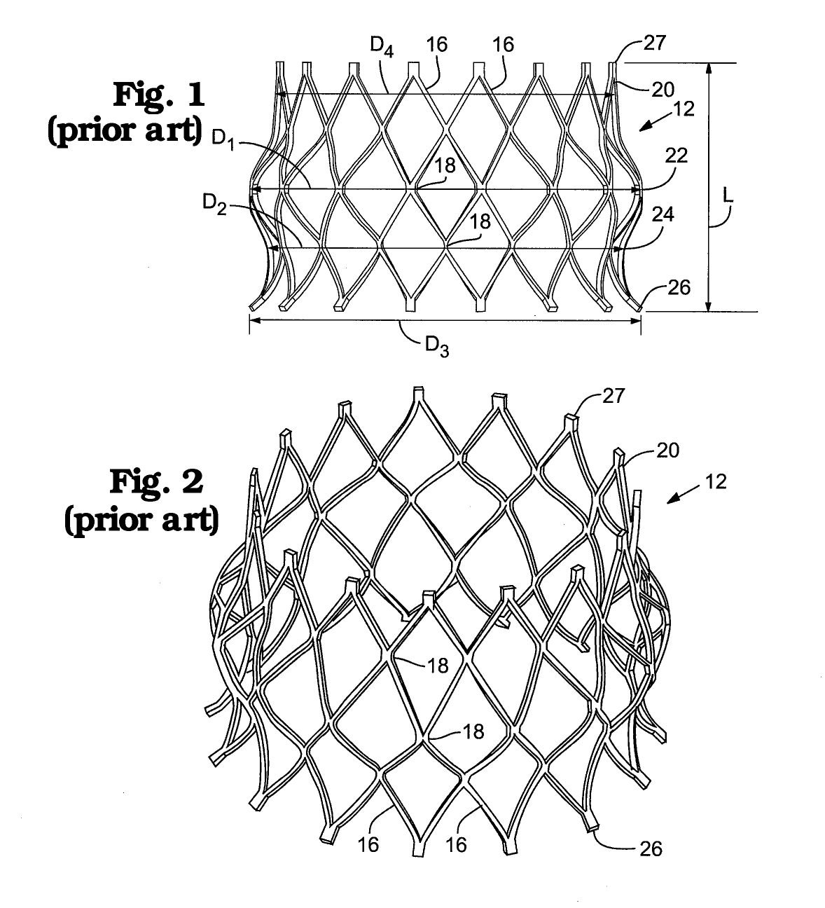

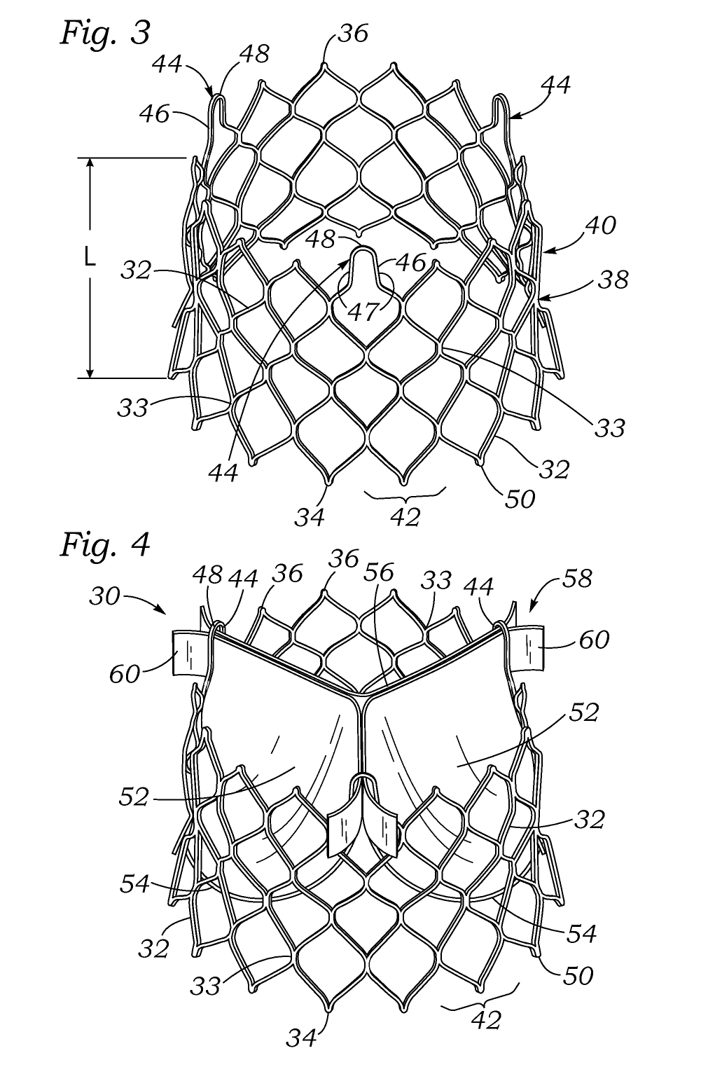

FIG. 3 shows one embodiment of an improved support frame 30. The support frame 30 can comprise a plurality of strut members 32 interconnected to each other to form a mesh structure having an inflow end 34 and an outflow end 36. In some embodiments, the mesh structure can have an overall curved shape that tapers inwardly from the inflow end 34 to a reduced diameter section 38, increases in diameter from the reduced diameter section 38 to a distended intermediate section 40, and then tapers from the intermediate section 40 toward the outflow end 36 of the mesh structure. The intermediate section 40 can be sized and shaped to extend into the Valsalva sinuses in the root of the aorta to assist in anchoring the valve in place once implanted. The frame 30 can have an overall shape similar to the frame 12 shown in FIGS. 1 and 2.

As shown, the support frame 30 can be formed from a plurality of longitudinally extending, generally sinusoidal shaped frame members, or struts, 32. The struts 32 are formed with alternating bends and can be welded or otherwise secured to each other at nodes 33 formed from the vertices of adjacent bends so as to form a mesh structure. In some embodiments, the frame 30 can be formed as a single, integral body. For example, in some embodiments the frame 30 can be laser cut from metal tubing as a single piece.

The strut members 32 of support frame 30 can be arranged to form repeating diamond-shaped, expandable cells 42, with multiple rows of such cells. In the embodiment shown in FIG. 3, the support frame 30 includes four rows of cells, although more or fewer rows of cells are also possible. A first row positioned adjacent the inflow end 34 and two intermediate rows can be formed of substantially identical cells 42 around the entire circumference of the support frame 30. A fourth row positioned adjacent the outflow end 36 can include three commissure tip cells 44 having a different shape than the diamond-shaped cells 42. The commissure tip cells 44 can be spaced around the circumference of the support frame 30 adjacent the outflow end 36, with diamond-shaped cells 42 positioned between the commissure tip cells 44. The commissure tip cells 44 can be positioned to correspond to the commissures of the leaflets (e.g., the points at which adjacent leaflets are secured together). Thus, in a prosthetic valve with three leaflets, the support frame 30 can include three angularly spaced commissure tip cells 44. In embodiments of a prosthetic valve with more or fewer leaflets, more or fewer commissure tip cells can be positioned in the support frame, respectively.

The commissure tip cells 44 can include an elongated projection 46 having two substantially straight, axially extending struts 47 joined at their upper ends by a rounded tip 48 adjacent the outflow end 36. By contrast, the diamond-shaped cells 42 terminate at an acute vertex 50 where adjoining strut members 32 come together. As shown, the projections 46 in the illustrated embodiment extend beyond the vertices 50 of the other cells at the outflow end 36 of the frame 30. The elongated projections 46 and rounded tips 48 of the commissure tip cells 44 allow for improved leaflet attachment as compared to prior art support frames, as will be discussed in more detail in connection with FIG. 4, below.

The struts 32 can comprise a suitable shape memory material, such as the nickel titanium alloy known as Nitinol, that allows the valve to be compressed to a reduced diameter for delivery in a delivery apparatus (suitable delivery apparatuses are described in the '040 Application) and then causes the valve to expand to its functional size inside the patient's body when deployed from the delivery apparatus. If the valve is a balloon-expandable valve that is adapted to be crimped onto an inflatable balloon of a delivery apparatus and expanded to its functional size by inflation of the balloon, the support frame 30 can comprise a suitable ductile material, such as stainless steel.

When the valve is in its expanded state the diameters of the distended intermediate section 40 and the inflow end 34 can be configured to be greater than the diameter than the native annulus in which the valve is to be implanted. In this manner, the overall shape of the support frame 30 can assist in retaining the valve at the implantation site. More specifically, the valve can be implanted within a native valve (the aortic valve in the illustrated example) such that the reduced diameter section 38 is positioned within the aortic annulus, the intermediate section 40 extends slightly above the aortic annulus into the Valsalva's sinuses, and the flared inflow end 34 extends slightly below the aortic annulus. The valve is retained within the native valve by the radial outward force of the reduced diameter section 38 against the surrounding tissue of the aortic annulus as well as the geometry of the support frame. Specifically, the intermediate section 40 and the flared inflow end 34 extend radially outwardly beyond the aortic annulus to better resist against axial dislodgement of the valve in the upstream and downstream directions (toward and away from the aorta). Depending on the condition of the native leaflets, the valve typically is deployed within the native annulus with the native leaflets folded upwardly and compressed between the outer surface of the support frame and the walls of the Valsalva sinuses.

Known prosthetic valves having a self-expanding frame typically have additional anchoring devices or frame portions that extend into and become fixed to non-diseased areas of the vasculature. Because the shape of the support frame 30 assists in retaining the prosthetic valve in place, additional anchoring devices are not required and the overall length L of the support frame 30 can be minimized to prevent the outflow end 36 from extending into the non-diseased area of the aorta, or to at least minimize the extent to which the outflow end 36 extends into the non-diseased area of the aorta.

As shown in FIG. 4, the prosthetic valve support frame 30 can include a plurality of leaflets 52 supported by the support frame 30. The plural leaflets 52 of the valve have respective inflow end portions 54 and outflow end portions 56. The inflow end portions 54 of the leaflets 52 can be secured to the inside of the support frame 30 near the inflow end portion 34 of the support frame. Suitable attachment methods for the inflow end portion of the leaflets are described and illustrated in the '040 Application, which is incorporated herein by reference. Generally, the prosthetic valve can include an annular reinforcing skirt that is secured to the outer surfaces of the inflow end portions of the leaflets at a suture line adjacent the inflow end of the valve. The inflow end portion of the leaflet assembly can be secured to the support frame by suturing the skirt to struts of the lower section of the support frame such that the skirt is sandwiched between the frame and the lower end portions of the leaflets.

The outflow end portions 56 of the leaflets 52 define angularly spaced commissures 58 corresponding to the points where adjacent respective leaflets 52 meet. Each leaflet 52 can include opposing leaflet tabs 60 at either end of the leaflet 52. Thus, two leaflet tabs 60 (e.g., one leaflet tab 60 from each of two adjacent leaflets 52) meet at each commissure 58. Each pair of leaflet tabs 60 can extend through a respective elongated projection 46 of a respective commissure tip cell 44. Each projection 46 serves as a commissure window frame portion through which the leaflet tabs 60 extend. After passing through the commissure tip cells 44, the leaflet tabs 60 can be sutured together. In this manner, the leaflets 52 can be coupled to one another such that the leaflet sutures are positioned outside of the support frame 30, and thus away from the high stress regions inside the support frame 30.

The leaflets can comprise any suitable biological material (e.g., pericardial tissue, such as bovine, porcine, or equine pericardium), other biological membranes, bio-compatible synthetic materials and fabrics, or other such materials, such as those described in U.S. Pat. No. 6,730,118, which is incorporated herein by reference.

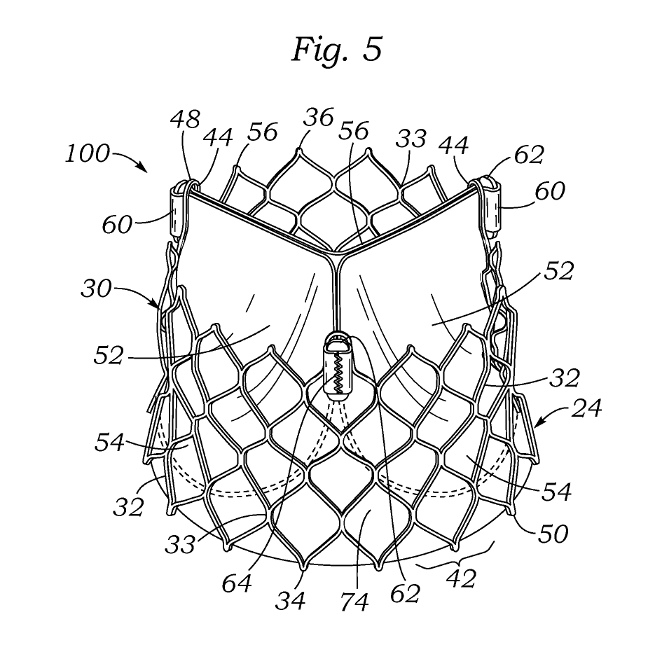

FIG. 5 shows one embodiment of a prosthetic valve 100, showing how the inflow end portions 54 and outflow end portions 56 of the leaflets 52 can be coupled to the support frame 30. Near the outflow end portions 56, the leaflet tabs 60 can be wrapped around, for example, a cloth covered pin, or wedge, 62. After passing through the commissure tip cells 44, in some embodiments, the leaflet tabs 60 can be sutured together around a small component, such as a cloth covered pin 62, a cloth covered Mylar insert, a thin polyester insert, or a small bar or pin made of metal and/or plastic. The bars or pins 62 can be of very small diameter and positioned outside of the support frame 30, as shown, so as not to limit the amount the support frame can be crimped for delivery. After being wrapped around the pin 62, the leaflet tabs 60 can be sutured to one another via one or more leaflet sutures 64. In some embodiments, the pin 62 (e.g., a cloth covered pin) can be coupled to the support frame 30, such as by a single suture, to ensure that the pin 62 is not dislodged from the leaflet tabs 60. For example, in some embodiments, at least a portion of the support frame 30 can be cloth-covered, and one or more sutures can secure the cloth covering of the bar or pin 62 to the cloth covering of the support frame 30.

Because the leaflet sutures 64 can be positioned outside of the support frame 30 and outside of a cloth covered pin or insert 62, the leaflet sutures can be positioned far enough away from the high stress regions of the leaflets so as to be substantially shielded from the stresses applied to the leaflets during physiologic loading. This positioning can advantageously allow for use of a thinner leaflet material (such as porcine pericardium), which in prior art frames may not be suitable for use due to the risk of leaflet sutures tearing through the leaflet material.

FIG. 5 also illustrates one embodiment of securing the inflow end portions 54 of the leaflets 52 to the support frame 30. For example, the prosthetic valve 100 can include an annular reinforcing skirt 74 that is secured to the outer surfaces of the inflow end portions 54 of the leaflets 52 along a suture line that tracks the lower curved edges of the leaflets. The annular reinforcing skirt 74 in turn can be secured to the support frame 30 by suturing the skirt 74 to the struts 32 of support frame 30. In some embodiments, the leaflet assembly can further include an inner reinforcing strip that is secured to the inner surfaces of the inflow end portions 54 of the leaflets 52. The skirt can comprise a suitable tear resistant fabric, such as PET or Dacron fabric.

In some embodiments, at least a portion of the support frame 30 and/or portions of the leaflet tabs 60 can be covered with a cloth covering, such as Dacron, in order to facilitate leaflet attachment. For example, the cloth covered pin 62 that the leaflet tabs 60 wrap around and/or the inflow end portion 54 of the leaflets 52 can be sutured to a cloth covering surrounding the support frame 30. In some embodiments, the commissure tip cells 44 can be cloth covered to facilitate leaflet attachment. In some embodiments, the entire support frame 30 can be cloth covered. In some embodiments, the leaflet tabs 60 can be reinforced with one or more reinforcing layers (e.g., fabric layers). U.S. Provisional Patent Application Nos. 61/472,083 and 61/386,833, which are incorporated herein by reference, disclose suitable materials and methods for applying a cloth covering to a stent frame such as support frame 30. Other techniques for securing leaflet tabs to commissure window frame portions outside of the lumen of the frame are disclosed in U.S. Provisional Patent Application No. 61/390,107, which is incorporated herein by reference. The techniques disclosed in App. 61/390,107 can be used to secure leaflet tabs 60 to frame portions 44.

Due to the geometry of the support frame, disclosed embodiments of an improved support frame for use with a prosthetic valve are particularly suited for replacing a non-stenotic aortic valve, which typically does not anchor a prosthetic valve as well as a calcified native valve. The stent desirably does not include additional anchoring devices or frame portions to assist in anchoring the valve in place. Consequently, the valve can be implanted without contacting non-diseased areas of the vasculature, which prevents or at least minimizes complications if future intervention is required. In alternative embodiments, disclosed support frames and prosthetic valves can be adapted to be deployed in native valves of the heart, or adapted to replace other valves within the body, such a venous valve.

The disclosed improved support frame and the overall prosthetic valve are radially compressible to a compressed state for delivery through the body to a deployment site and expandable to its functional size at the deployment site. Apparatuses particularly suited for percutaneous delivery and implantation of a self-expanding or balloon-expandable valve are described in detail in the '040 Application, which is incorporated herein by reference. Generally, the prosthetic valve can be implanted in a retrograde approach where the valve, mounted in a crimped state at the distal end of a delivery apparatus, is introduced into the body via, for example, the femoral artery and advanced through the aortic arch to the heart, as further described in U.S. Patent Publication No. 2008/0065011, which is incorporated herein by reference.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

I claim:

1. A prosthetic heart valve for implantation at an implantation site having an annulus, the valve comprising:
a radially expandable and compressible support frame comprising a plurality of strut members interconnected to each other to form a mesh structure comprising an inflow end, an outflow end, and a plurality of rows of expandable cells; and
plural leaflets having respective inflow end portions and outflow end portions, the outflow end portions of the leaflets defining angularly spaced commissures and comprising opposing leaflet tabs positioned at first and second ends of the leaflets, the leaflets being arranged such that the leaflet tabs of adjacent leaflets are aligned to form a pair of leaflet tabs at each commissure, and wherein each pair of leaflet tabs extends through a respective expandable cell in one of the rows of expandable cells.

2. The heart valve of claim 1, wherein the plurality of rows of cells includes an outflow row at the outflow end of the frame, some of the cells of the outflow row comprising elongated projections, and each pair of leaflet tabs extends through the elongated projections.

3. The heart valve of claim 1, wherein the elongated projections extend beyond the ends of adjacent cells in the outflow row.

4. The heart valve of claim 1, wherein the leaflets comprise porcine pericardium.

5. The heart valve of claim 1, further comprising at least one leaflet suture securing each pair of leaflet tabs together, the leaflet sutures being positioned outside of the support frame.

6. The heart valve of claim 1, further comprising at least one bar or pin positioned outside of the support frame and adjacent to each commis sure, such that each pair of leaflet tabs is configured to wrap around the bar or pin.

7. The heart valve of claim 6, wherein the bars or pins are cloth covered and secured to the support frame via one or more pin sutures positioned outside of the support frame.

8. The heart valve of claim 1, wherein at least a portion of the support frame is cloth covered.

9. The heart valve of claim 1, wherein the mesh structure comprises a distended intermediate portion having a first diameter at a first location, the intermediate portion tapering in a direction toward the inflow end to form an inflow end portion having a second, smaller diameter at a second location.

10. The heart valve of claim 1, wherein the inflow end portion of the mesh structure has a flared portion that increases in diameter from the second location to a third location at the inflow end of the mesh structure having a third diameter, the first and third diameters being greater than the second diameter.

11. The heart valve of claim 1, wherein the intermediate portion tapers in a direction toward the outflow end to form an outflow end portion having a fourth diameter that is less than the first diameter.

12. The heart valve of claim 1, wherein the inflow end portions of the leaflets are secured to the inside of the support frame near the inflow end portion of the support frame.

13. A support frame for a prosthetic heart valve comprising:
a radially expandable and compressible frame comprising a plurality of strut members interconnected to form a mesh structure having an inflow end and an outflow end;
wherein the strut members form expandable cells and the frame comprises a plurality of rows of cells; and
wherein the row adjacent the outflow end includes a plurality of commissure tip cells having an elongated projection formed from two substantially straight axially extending struts joined by rounded tip.

14. The support frame of claim 13, wherein the mesh structure has an overall curved shape that tapers inwardly from the inflow end to a reduced diameter section, increases in diameter from the from the reduced diameter section to a distended intermediate section, and then tapers from the intermediate section toward the outflow end of the mesh structure.

15. The support frame of claim 13, wherein the strut members are formed of shape memory material.

16. The support frame of claim 13 having three commis sure tip cells spaced around a circumference of the support frame adjacent the outflow end.

17. The support frame of claim 13, wherein the frame is formed as a single, integral body.

18. The support frame of claim 17, where the frame is laser cut from a piece of metal tubing.

19. The support frame of claim 13, wherein at least a portion of the frame is covered with cloth.

20. The support frame of claim 13, wherein the projections extend in an outflow direction beyond the cells in the row of cells adjacent the outflow end of the frame.
